(19) United States
(12) Patent Application Publication
(10) Pub. No.: US 2004/0157308 A1
(43) Pub. Date: Aug. 12, 2004

(54) Alpha-galactosidase as food-grade genetic marker

(76) Inventors: Sylvain Moineau, Notre-Dame-du-Bon-Conseil (CA); Isabelle Boucher, Quebec (CA)

Correspondence Address: OGILVY RENAULT, 1981 MCGILL COLLEGE AVENUE, SUITE 1600, MONTREAL, QC, H3A2Y3, CA

(21) Appl. No.: 10/471,601

(22) PCT Filed: Apr. 5, 2002
(86) PCT No.: PCT/CA02/00462

(51) Int. Cl. C12N 9/40; C12Q 1/68; C07H 21/04; C12N 15/74

(52) U.S. Cl. 435/208; 435/6; 435/69.1; 435/320.1; 536/23.2; 435/252.3

(57) ABSTRACT

The invention relates to a 4007 bp DNA fragment from the strain Lactococcus raffinolactis ATCC 43920 containing two genes. The first gene (named aga) codes for an enzyme with an alpha-galactosidase activity. The second gene (named galR) codes for a transcriptional regulator which would act as a regulator of aga. When present in a lactic acid bacterium such as Lactococcus lactis, this DNA fragment can modify the sugar fermentation profile of the strain from melibiose-negative to melibiose-positive. The utilisation of a culture media containing melibiose as the sole carbon source and bromcresol purple as pH indicator allows the identification of the melibiose-fermenting bacteria as yellow colonies on a purple background.

### BACKGROUND OF THE INVENTION

(a) Field of the Invention

The invention relates to genetic modification of microorganisms, more particularly to lactic acid bacteria used in various foods. The invention also relates to DNA constructions encoding a selectable marker other than an antibiotic resistance marker, vectors and/or cells including the constructs. The present invention also relates to probiotic microorganisms improving the digestion of certain foods and preventing gastrointestinal problems and other symptoms associated with these foods.

(b) Description of Prior Art

Theoretically, it should be possible to improve the activity of the recombinant protein by increasing its level of expression (Froseth et al., 1991, J. Dairy Sci. 74:1445-1453). This could be done either by providing many copies of the gene encoding the relevant enzyme(s) and/or by genetically engineering the gene so that it is linked to an agent, such as a promoter, providing for high expression. However, in order to assess the success of such genetic engineering it is necessary to include in the engineering process selectable markers in order to identify successfully transformed cells suitable to use as enzymes. Markers that are currently used are antibiotic resistance markers and the tests for successful transformation typically involve exposure of a cell population to a selected antibiotic and subsequent isolation of the cells showing antibiotic resistance.

Food-grade vectors for Lactococcus lactis including selectable markers were previously described. Some of these markers are dominant while other are based on the complementation of mutants with a specific deficiency. These markers can be used to select transformed cells using adapted culture media.

Dominant Markers

A dominant marker based on the nisin resistance gene was used in various applications. A system based on Lactococcus lactis cadmium resistance gene was also proposed as a food grade marker, alone or in association with the nisin resistance gene. Media containing nisin and/or cadmium were used to identify the transformed cells.

Another dominant marker for L. lactis was based on Pediococcus pentosaceus scrA/scrB genes that code for sucrose transport and hydrolysis (Leenhouts et al., 1998, Appl. Microbiol. Biotechnol. 49:417-423). Transformants were selected on medium containing sucrose as the sole fermentation substrate.

Complementation Markers

Lactose-negative Lactococcus lactis mutants can become lactose-positive by supplying missing functions. Some lactose-negative mutants contain a defective lacF gene, coding for the Enzyme IIA of the lactose PTS, and thus can be complemented with the wild-type lacF gene.

This genetic addition restores their ability to grow on a medium containing lactose as the sole fermentation substrate.

The L. lactis thymidilate synthase gene thyA was reported as a potential complementation marker that could be used in L. lactis but no L. lactis thyA deficient strains were yet reported. In Rhizobium meliloti, thyA⁻ mutants are complemented with the marker and selected for their ability to grow in absence of thymine and thymidine.

Another food-grade cloning vector using an amber suppressor (supD) as selectable marker is known for over-expressing a variety of genes in industrial strains of Lactococcus lactis. Using suppressible pyrimidine auxotrophic mutants, only the cells containing the amber suppressor were complemented and selected in pyrimidine-free medium such as milk.

This extensively used test for determining successful transformation cannot be employed in the production of different products for human or animal use. This is because incorporating a gene conferring antibiotic resistance into a product could be potentially hazardous for a number of reasons. An adverse reaction, as allergy for example, can occurred in an unusually foreign product as an antibiotic, the antibiotic could not be administered to the individual to clear the product. Also, it is of concern that the antibiotic resistance gene could be transferred to other microorganisms making them no longer amenable to controlling the antibiotic.

Thus, there is a need to find a safe selectable marker and by this term we mean a marker that can be used in a system, or a cell that will eventually be given to animals and to certain humans.

Probiotics

The ingestion of certain food by mammals results in flatulence and/or other gastrointestinal symptoms. Certain food is extremely flatugenic as milk and milk products, legumes (e.g., peanuts, beans), some cruciferous vegetables (e.g., cabbage, brussels sprouts) and certain fruit (e.g., raisins, bananas, apricots). The principal reason why the previously mentioned food causes flatulence is the body's inability to digest certain carbohydrates contained within these products. The mammalian inability to digest those carbohydrates allows putrefactive bacteria in the large intestine to break down the carbohydrates by fermentation. This results in the formation of excessive levels of rectal gas, primarily carbon dioxide, methane and hydrogen.

The mammalian ability or inability to digest certain carbohydrates depends upon the presence or absence of certain enzymes in the digestive system and the type of carbohydrate to be digested. For example, the human's ability to secrete specific enzymes enabling the digestion of the carbohydrate, lactose (commonly called “milk sugar”), depends upon a number of factors, e.g., age, race and health. Beta-D-galactoside-galactohydrolase (commonly called “beta-galactosidase” or “lactase”) is secreted within a human's digestive system in order to hydrolyze lactose (a molecule which contains the beta-galactoside linkage) into digestible monosugars, glucose and galactose. When beta-galactosidase is not enough active in order to hydrolyze lactose, in vitro treatment of milk or oral administration of microbial beta-galactosidase(s) for in vivo use duplicates the function of the naturally occurring neutral intestinal beta-galactosidase found on the gut wall (known as intestinal lactase).

In vitro treatment of milk with beta-galactosidase was first performed by the consumer at home. Approximately ten years ago, in vitro treatment of milk was done on a commercial scale by the dairy industry. Since approximately 1984, a beta-galactosidase preparation has been available on a substantial scale by a number of companies, including Lactaid Inc., for in vivo use.

The success of an ingestible form of beta-galactosidase for in vivo use was not entirely surprising, since the ingested enzyme structurally and functionally duplicates beta-galactosidase existing within the human digestive system. There was initial concern as to whether an ingested form of beta-galactosidase subject to varying pH levels would operate effectively on the human stomach and/or intestine. The fact that certain dosages of oral beta-galactosidase preparations did indeed substantially digest dietary lactose in the stomach and small intestine of people lacking natural form of this enzyme showed that at least some enzymes from microbial sources were not inactivated by the conditions of acidity, protein digestion, temperature or motility found in the gastrointestinal tract.

The lactose of milk and milk products is digestible by essentially all mammals during at least parts of their lives. But this is not the case with certain sugars contained in vegetables and certain fruits. The above-mentioned flatugenic vegetables and fruit contain one or more of the carbohydrates: raffinose, stachyose and verbascose. What these three oligosaccharide molecules all have in common is a D-galactose sugar linked to another sugar unit via an alpha-galactoside linkage.

Enzymes of the class alpha-D-galactoside-galactohydrolase (commonly called “alpha-galactosidase”) have the capacity to hydrolyze this alpha-galactoside sugar linkage. D-galactose is a monosaccharide which can be absorbed by the intestinal cell into the body and thereafter converted to glucose. Humans and other mammals cannot digest the three oligosaccharides to liberate D-galactose, since their digestive systems do not produce alpha-galactosidase.

In vitro use of alpha-galactosidase to make the previously-mentioned oligosaccharides digestible is well known. The U.S. Pat. Nos. 3,966,555; 4,241,185; and 4,431,737 disclose methods for producing and/or stabilizing alpha-galactosidase by culturing various microorganisms. All these patents disclose or imply is that alpha-D-galactosidase can be used in vitro in food processing and/or by addition to foodstuffs for a period of up to 12 hours. This demonstrates the ability to hydrolyze, in vitro alpha-D-galactoside-linked sugars.

Alpha-galactosidase is generally provided in powder form and may be combined with one or more excipients, which are also in powder form, to produce solid forms of the ingestible composition, i.e., tablet, capsule, powder. Concentrated (highly pure) liquid alpha-galactosidase may be formed into an ingestible powder composition thus, a liquid form of alpha-galactosidase is absorbed and/or adsorbed by dry powder excipient(s), diluted and evenly dispersed throughout the tablet or capsule preblended. Liquid forms of alpha-galactosidase can also be taken orally in soft-gel capsule form, or administered in drop or spoon size doses from a bottle; or for incorporation directly in the food just before eating. In such cases, the liquid is diluted with other appropriate diluent liquids or excipients. The degree of dilution will depend on the use intended; very little dilution for liquid gel capsule and substantial dilution for preprandial addition directly into food. However, this method needs a high control of the quality during the production processes, and addition of the enzyme in the food products at different step of preparation depending on the product.

The need for methods and systems allowing synthesis and delivery of digestion modulating molecules directly in food products or in vivo in the intestinal tract after oral ingestion is still there. The system should be food-grade proof.

### SUMMARY OF THE INVENTION

One object of the present invention is to provide food-grade cloning vector and bacteria containing it, which comprise DNA sequence coding for the alpha-galactosidase isolated from Lactococcus raffinolactis.

Another object of the present invention is to provide a cloning vector further comprising the natural alpha-galactosidase regulator, which will provide an increased stability of a vector carrying this regulator sequence in a transformed host bacteria.

In accordance with the present invention there is provided an isolated alpha-galactosidase protein comprising amino acid sequence as set forth in SEQ ID NO:1, fragments or analogs thereof, having alpha-galactosidase activity.

In accordance with the present invention there is also provided an isolated alpha-galactosidase regulator protein comprising amino acid sequence as set forth SEQ ID NO:2, fragments or analogs thereof having an alpha-galactosidase regulator activity.

In accordance with the present invention there is provided an isolated DNA sequence as set forth in SEQ ID NO:3, SEQ ID NO:4, and SEQ ID NO:5.

In accordance with the present invention there is provided an isolated DNA sequence from Lactotoccus raffinolactis selected from the group consisting of SEQ ID NO:3, SEQ ID NO:4, and SEQ ID NO:5.

In accordance with the present invention there is provided a vector suitable for transforming a host cell, the vector comprising;

a DNA sequence as in claim 6;

a suitable promoter allowing expression of the DNA sequence in the host cell;

wherein the DNA sequence encodes for a protein having alpha-galactosidase activity

The vector may further comprise a DNA sequence as set for in SEQ ID NO:5, and coding for an alpha-galactosidase regulator, and may be expressed in the host cell as a selectable marker. The selectable marker may be used as a food-grade vector.

In accordance with the present invention there is also provided a host cell transformed with vector described herein, and is a food-grade host cell

The host cell may be selected from the group consisting of animal cell, yeast, and bacteria.

The bacteria may be selected from the group consisting of Lactococcus, Streptococcus, Lactobacillus, Leuconostocs, Pediococcus, Bifidobacterium, Oenococcus, and Propionibacterium.

In accordance with the present invention there is also provided a method of modulating intestinal digestion in a subject comprising the step of administrating orally to a subject a cell expressing of at least one of alpha-galactosidase or alpha-galactosidase regulator. The cell of this method can be a wild type cell, as for example but not limited to Lactococcus rafinolactis, or a transformed cell allowing the expression of at least one of alpha-galactosidase or alpha-galactosidase regulator.

The subject may be a human, a mammal or a bird.

The host cell used to perform the method of the invention may be selected from the group consisting of yeast, mould, and bacteria.

In accordance with the present invention there is also provided a method of modulating intestinal digestion in a subject comprising administrating orally to a subject a composition comprising alpha-galactosidase protein, a fragment or an analog thereof having an alpha-galactosidase activity.

Another object of the present invention is the use of at least one of an alpha-galactosidase or alpha-galactosidase regulator in the preparation of a composition for modulating intestinal digestion in a subject; the use of a host cell transformed with a food-grade vector allowing expression of at least one of alpha-galactosidase or alpha-galactosidase regulator in the preparation of a composition for modulating intestinal digestion in a subject; or the use or a DNA sequence as defined in claims 3 to 5 in the preparation of a vector allowing expression of at least one of an alpha-galactosidase protein or an alpha-galactosidase regulator.

For the purpose of the present invention the following terms are defined below.

The term “antibiotic” as used herein is intended to mean any of various chemical substances such as penicillin, ampicillin, streptomycin, neomycin or tetracycline produced by various microorganisms, or their synthetic counterparts.

The expression “food-grade label” as used herein is refers to food products that have been approved by regulatory authorities as being safe, and acceptable for consumption by animals and human.

### BRIEF DESCRIPTION OF THE DRAWINGS

FIG. 1 illustrates the genetic organization of the 4007 bp genomic DNA fragment encoding the alpha-galactosidase (aga) and its regulator (galR);

FIG. 2 illustrates genotypic and phenotypic analysis of galA-mutants;

FIG. 3 illustrates the genetic organization of the plasmid pRAF800;

FIG. 4 illustrates the map of the plasmid pRAF800; and

FIG. 5 illustrates the expression profile of the plasmid pRAF800.

### DETAILED DESCRIPTION OF THE INVENTION

Food-Grade Selectable Marker

In accordance with the present invention, new genetic marker for selecting bacteria transformed for different applications, without using antibiotic resistance as selectable marker is provided.

Lactic acid bacteria play a very important role in a large number of food fermentation processes. The fermentation processes in which lactic acid bacteria play an important role do not only include fermentation of milk, resulting in products like yogurt, sour cream and cheese, but also includes fermentation of meat, fish, fruit, vegetables, beans and cereal products.

The role of lactic acid bacteria is to make these fermented products microbiologically more stables and to improve the taste and palatability of these products. Fermented food products containing certain types of lactic acid bacteria are also important in the development of new products that have a positive impact on the health of the consumers. Consequently lactic acid bacteria are of large economic importance. It is known that genetic properties, that are important to ensure that lactic acid bacteria perform the right type of fermentation, are located on extrachromosomal DNA, and most often called plasmids. Plasmids have the advantage that they exist normally in the cell in multimeric form, which also means that a certain gene located on such a plasmid exists in the cell in multicopy form, which may result in a higher expression of the proteins encoded by these genes.

In one embodiment of the present invention, a 4007 bp HindIII/EcoRI genomic DNA fragment isolated from the strain Lactococcus raffinolactis ATCC 43920 is provided. This fragment contains two genes, named herein aga and galR, endocing respectively for an alpha-galactosidase enzyme and its repressor. The aga gene codes for a protein of 735 amino acids with an alpha-galactosidase activity (SEQ ID NO:1). This enzyme hydrolyzes alpha-galactosides such as raffinose and melibiose, releasing the alpha-galactose moiety of the sugar.

In another embodiment of the present invention, there is provided a DNA sequence encoding the alpha-galactosidase repressor, and galR of L. raffinolactis.

The galR gene codes for a protein of 345 amino acids similar to members of the GalR family of transcriptional regulators (SEQ ID NO:2). GalR is believed to act as a transcriptional repressor of aga.

Melibiose (6-O-α-D-galactopyranosyl-D-glucose) is a disaccharide obtained from raffinose by fermentation with a yeast. Melibiose is not commonly fermented by most lactic acid bacteria. However, this sugar is hydrolysed by the alpha-galactosidase of the present invention, into galactose and glucose, which are normally metabolised by a wide variety of lactic acid bacteria.

In one embodiment of the present invention, a 4007 bp DNA fragment that can be used as a dominant in cloning techniques commonly used in molecular biology laboratories is provided. It can also pretend to a food-grade label allowing its use for the genetic modification of lactic acid bacteria.

The 4007 bp DNA fragment comprising galR and aga can modify the fermentation pattern of lactic acid bacteria from melibiose-negative to melibiose-positive. When associated with a functional plasmid replication module, it can be transferred into various host strains. The modified bacteria are identified as melibiose-fermenting yellow colonies formed on solid media containing melibiose as the only carbon source and bromcresol purple as the pH indicator. The presence of GalR may enhance the stability of the genetic construction by regulating the expression of aga by the cell.

The 4007 bp DNA fragment can be used as a dominant genetic marker in cloning techniques commonly used in molecular biology laboratories.

In another embodiment of the invention, when introduced into a melibiose-negative bacteria as Lactococcus lactis, the DNA fragment encoding for the α-galactosidase confers the ability to metabolise the melibiose.

In still another embodiment of the invention, a molecular tag for strain identification based on the melibiose fermentation phenotype conferred by the 4007 DNA fragment is provided.

In still another embodiment of the invention, a molecular tag for differential enumeration in mixed cultures is provided.

Still in another embodiment, the DNA fragment encoding for the α-galactosidase was isolated from the bacteria Lactococcus raffinolactis that is not currently used in the dairy industry.

In the DNA construct of the invention, the analogue of the DNA sequence encoding a polypeptide having an alpha-galactosidase activity may, for instance, be a subsequence of the DNA sequence, a genetically engineered modification of the sequence which may be prepared by different procedures, e.g. by site-directed mutagenesis, and/or a DNA sequence with substantial similarity to the alpha-galactosidase having the amino acid sequence shown in SEQ ID NO:1.

According to one embodiment of the present invention, the sequence of the analogues is important as long as the analogue has at least one of the properties, i.e. that the hybridization of a DNA sequence with the DNA sequence shown in the SEQ ID NO:3 or SEQ ID NO:4 or SEQ ID NO:5 or with a suitable oligonucleotide probe prepared on the basis of the DNA sequences or on the basis of the polypeptide shown in SEQ ID NO:1 or SEQ ID NO:2, may be carried out under any suitable conditions allowing the DNA sequences to hybridiz; the immunological cross reactivity may be assayed using an antibody raised against or reactive with, at least one epitope of the alpha-galactosidase enzyme comprising the amino acid sequence shown in SEQ ID NO:1. The antibody, which may either be monoclonal or polyclonal, may be produced by methods known in the art. The immunological cross-reactivity may be determined using assays known in the art, examples of which are Western Blotting or radial immunodiffusion assay. It is believed that an identity of above 50% such as above 80%, and in particular above 95% with the amino acid sequence shown in SEQ ID No:1 is indicative for homology with the alpha-galactosidase encoded by the DNA sequences shown in SEQ ID NO:3, SEQ ID NO:4 or SEQ ID NO:5. As far as the present inventors are aware that this is the only alpha-galactosidase with a known amino acid sequence that show any comparable identity to the alpha-galactosidase encoded by the DNA construct of the invention; or another property is that the sequence of an analog may be determined by comparing the amino acid sequences of the polypeptide encoded by the analogue and the polypeptide sequence shown in SEQ ID NO:1 by use of algorithms. In the present context, “identity” is used in its conventional meaning, i.e. intended to indicate the number of identical amino acid residues occupying similar positions in the two (or more) amino acid sequences to be compared. The identity can be between about 40 to 100 percents if the activity of the analog is the same as this one of the alpha-galactosidase disclosed in the application.

The DNA sequence may, for instance, be isolated by establishing a DNA or genomic library from an organism expected to harbor the sequence, e.g. a cell of any of the origins mentioned above, and screening for positive clones by conventional procedures. Examples of such procedures are hybridization to oligonucleotide probes synthesized on the basis of the full or partial amino acid sequence of the L. raffinolactis alpha-galactosidase comprising the amino acid sequence shown in SEQ ID NO:1 in accordance with standard techniques, and/or selection for clones expressing an appropriate biological activity as defined above, and/or selection for clones producing a protein which is reactive with an antibody raised against the L. raffinolactis alpha-galactosidase.

A method for isolating a DNA construct of the invention from a DNA or genomic library is by use of polymerase chain reaction (PCR) using degenerate oligonucleotide probes prepared on the basis of the nucleic acid sequence shown in SEQ ID NO:3. For instance, the PCR may be carried out using the techniques described in the U.S. Pat. No. 4,683,202, the entire content of which is hereby incorporated by reference.

Alternatively, the DNA sequence of the DNA construct of the invention may be prepared synthetically by different established methods. According to the phosphoamidite method, oligonucleotides are synthesized, e.g. in an automatic DNA synthesizer. It may be then purified, annealed, ligated and cloned in appropriate vectors.

In another embodiment of the present invention, the DNA construct, or vector, may be made with mixed genomic and synthetic, or fragments thereof, the fragments corresponding to various parts of the entire recombinant DNA molecule.

As stated above, the DNA construct of the invention may also comprise a genetically modified DNA sequence. Such sequence may be prepared on the basis of a genomic or DNA sequence of the invention, suitably modified at a site corresponding to the site(s) of the polypeptide at which the introduction of the amino acid substitutions is desired, e.g. by site-directed mutagenesis using synthetic oligonucleotides encoding the desired amino acid sequence for homologous recombination in accordance with different procedures, for example but not limited to, by use of random mutagenesis, e.g. through radiation or chemical treatment.

Examples of suitable modifications of the DNA sequence are nucleotide substitutions which do not give rise to another amino acid sequence of the polypeptide, but which may correspond to the codon usage of the host organism into which the recombinant DNA molecule is introduced (i.e. modifications which, when expressed, results in e.g. an alpha-galactosidase comprising the amino acid sequence as shown in the appended SEQ ID NO:1), or nucleotide substitutions which do give rise to a amino acid sequence substantially identical to the appended SEQ ID NO:1, impairing properties of the polypeptide such as enzymatic properties thereof. Other examples of possible modifications are insertion of one or more nucleotides into the sequence, addition of one or more nucleotides at either end of the sequence and deletion of one or more nucleotides at either end of or within the sequence.

According to one embodiment of the present invention, the recombinant cloning vector carrying the DNA construct of the invention may be any vector that may conveniently be subjected to recombinant DNA procedures, and the choice of vector will often depend on the host cell into which it is to be introduced. Thus, the vector may be an autonomously replicating vector, i.e. a vector that exists as an extrachromosomal entity, the replication of which is independent of chromosomal replication, e.g. a plasmid or a bacteriophage. Alternatively, the vector may be one which, when introduced into a host cell, is integrated into the host cell genome and replicated together with the chromosome(s) into which it has been integrated.

In the vector, the DNA sequence should be operably connected to a suitable promoter sequence. The promoter may be any DNA sequence that shows transcriptional activity in the host cell of choice and may be derived from genes encoding proteins either homologous or heterologous to the host cell.

The cloning vector of the invention may also comprise a suitable terminator operably connected to the DNA construct of the invention. The terminator is suitably derived from the same source as the promoter of choice.

The vector may further comprise a DNA sequence enabling the vector to replicate in the host cell in question. Examples of such sequences are the origins of replication of plasmids pUC19, pACYC177, pUB110, pE194, pAMB1 and pIJ702.

While intracellular expression may be advantageous in some respects, e.g. when using certain bacteria as host cells. In order to obtain extracellular expression, the cloning vector should normally further comprise a DNA sequence encoding a preregion, i.e. a signal peptide, permitting secretion of the expressed alpha-galactosidase or a variant thereof into the cultured medium.

The procedures used to ligate the DNA construct of the invention, the promoter, terminator and other elements, including the repressor galR to insert them into suitable vectors containing the information necessary for replication, are well known to the scientists.

In a yet further embodiment, the present invention relates to a method for producing a polypeptide of the invention, which method comprises cultivating a host cell under suitable conditions allowing the production and recovering of the alpha-galactosidase from the cells and/or culture medium.

It may be suitable to produce substantially pure alpha-galactosidase or alternatively alpha-galactosidase preparation free from certain undesired enzymatic side-activities (an example of which—for some uses of the alpha-galactosidase—is invertase) one may either remove the side-activity(ies) by purification or one may choose a production organism incapable of producing the side-activity(ies) concerned.

The alpha-galactosidase encoded by the DNA construct of the invention may be used for a number of purposes involving hydrolysis of alpha-galactosides.

The presence of the α-galactosidase alone is sufficient to obtain a melibiose-positive phenotype, however the presence of the regulator may increase the long-term stability of the phenotype in bacterial strains.

Probiotic

Ingestion of a composition comprising an effective amount of alpha-galactosidase in a non-toxic ingestible excipient, substantially simultaneous or contemporaneous with the ingestion of foods containing alpha-D-galactoside-linked sugars, results in the complete or partial hydrolysis of these oligosaccharides into their simplest absorbable constituents, in vivo. The time period for ingesting the alpha-galactosidase containing composition is preferably from about ¼ hour before to about ¼ hour after ingestion of foods containing the alpha-D-galactoside-linked sugars. Effectiveness can be expected to decrease appreciably with increasing time displacement of the alpha-galactosidase ingestion from the time of the meal because, to be effective, the enzyme must mix in the stomach with the ingested food, so they must be ingested more or less simultaneously. The most appropriate time to ingest the alpha-galactosidase-containing composition is simultaneous with the alpha-D-galactoside-linked sugars-containing foods.

The enzyme can be delivered in the form of a tablet, soft-gel capsule or similarly shaped pill in ingestible form, although plain liquid can be used as mentioned earlier. Also, a powder form of the ingestible composition which is packaged or kept on the table in a “salt-shaker” can be sprinkled on the food, or a liquid form, such as that administered from a bottle, or mixed with the food immediately before eating. Such immediate prior mixing is not an in vitro use, but a version of in vivo use, with “immediate” meaning any time from “in the plate on the table” to several hours prior mixing, since the enzyme activity will be in vivo, not in vitro, in any solid food.

Oral administration is just one way of supplying the enzyme to the digestive system. The ingestible composition could be administered through a tube or similar device that is connected to the stomach or small intestine. Furthermore, this invention is suited for various types of mammals and is not just limited for the use of humans. For example, one may find this invention particularly suited for pets, such as dogs or cats, which often experience symptoms and emit noxious odors associated with flatulence after they have ingested alpha-D-galactoside-linked sugar-containing foods.

In another embodiment of the present invention, food-grade bacteria synthesizing the α-gal of the invention, may be under living form and produce the α-gal enzyme directly into the digestive tract, the intestine, or in the blood of a subject.

### EXAMPLE I

### Food-Grade Plasmid Vector Based on Melibiose Fermentation for the Genetic Engineering of Lactococus lactis

Material and Methods

Bacterial Strains, Phages and Plasmids

Bacterial strains and plasmids used in this study are listed in Table 1. E. coli was grown in LB at 37° C., Lactococcus in M17 (Quelab, Montréal, Québec, Canada) at 30° C. supplemented with the appropriate sugar and P. acidilactici in MRS (Merck, Darmstadt, Germany) at 37° C. Carbohydrate fermentation was tested in BCP medium (2% tryptone, 0.5% yeast extract, 0.4% NaCl, 0.15% Na-acetate, 40 mg/l purple bromocresol). Enrichment of Mel+transformants was usually performed in liquid EL1 medium (1% tryptone, 0.4% NaCl, 0.15% Na-acetate, 40 mg/l purple bromocresol). Sugars were filter-sterilized and added to a final concentration of 0.5% to autoclaved media. The BCP and EL1 formulations were based on the Elliker medium (Elliker et al., 1956, J. Dairy Sci., 39:1611-1612). When required, antibiotics were added as follows: for E. coli, 50 μg/ml of ampicillin; for L. lactis, 5 μg/ml of erythromycin or chloramphenicol. All the antibiotics were purchased from Sigma-Aldrich (Oakville, Ontario, Canada). Phages were amplified on their respective L. lactis hosts. Phage sensitivity was determined by spot test (Moineau et al., 1992, Can. J. Microbiol., 38:875-882).

DNA Techniques

Routine DNA manipulations were carried out according to standard procedures. Restriction enzymes, alkaline phosphatase, RNAse free DNAse, RNAse Inhibitor (Roche Diagnostics, Laval, Québec, Canada), and T4 DNA ligase (Invitrogen Life Technologies, Burlington, Ontario, Canada) were used according to the supplier's instructions. All primers used were obtained from Invitrogen Life Technologies. Transformation of E. coli, L. lactis and P. acidilactici were performed as described below. Plasmid DNA from E. coli and L. lactis was isolated as previously described (Emond et al., 2001, Appl. Environ. Microbiol. 67:1700-1709). Lactococcus raffinolactis total DNA was isolated from 200 ml of an overnight culture in GM17 at 30° C. Pelleted cells were resuspended in 10 ml of lysis solution (6.7% sucrose, 50 mM Tris-HCl pH 8, 1 mM EDTA pH 8, lysozyme 30 mg/ml), and incubated at 37° C. for 20 min. Then, 1.12 ml of 10% SDS was added and the mixture was incubated at 60° C. for 10 min. After addition of 80 μl of proteinase K (20 mg/ml; Roche Diagnostics), the lysate was incubated at 60° C. for an additional 20 min. DNA was precipitated with 1/10 volume of 3M potassium acetate pH 7 and 2 volumes of 95% ethanol after 3 phenol:chloroform (1:1) extractions. The DNA precipitate was washed with 70% ethanol, air dried, and dissolved in 1 ml of ddH₂O containing RNAse A (5 μg/ml).

TABLE 1
Bacterial strains, phages, and plasmids used in this study

| Bacterial strain, | |
|---|---|
| phage, or plasmid | Relevant characteristics^{a} |
| Strains | |
| E. coil DH5α | supE44 Δlac U169 (f80 lacZΔM15) hsdR17 |
| | recA1 endA1 gyrA96 thi-1 relA1 |
| L. lactis subsp. cremoris | |
| MG1363 | Laboratory strain, plasmid free, Mel− |
| SMQ-741 | Industrial strain, Mel− |
| L. lactis subsp. lactis | |
| IL1403 | Laboratory strain, plasmid free, Mel− |
| SMQ-561 | Industrial strain, Mel− |
| Lactococcus raffinolactis | |
| ATCC43920 | Plasmid free, Mel+ |
| Streptococcus | Industrial strain, Mel− |
| thermophilus SMQ-301 | |
| Pediococcus acidilactici | |
| SMQ-249 | Industrial strain, Mel− |
| Phages | |
| c2 | c2 species, infects L. lactis MG1363 |
| p2 | 936 species, infects L. lactis MG1363 |
| Q37 | 936 species, infects L. lactis SMQ-741 |
| Plasmids | |
| pBS | Cloning vector for DNA sequencing, Ap^{r} |
| pGhost4 | Integration vector, Ts, Em^{r} |
| pNC1 | Replicon-screening vector, Ap^{r}, Cm^{r} |
| pNZ123 | Shuttle cloning vector, Cm^{r} |
| pTRKH2 | Shuttle cloning vector, Em^{r} |
| pGalA2 | pGhost4 + L. lactis MG1363 truncated galA, |
| | Ts, Em^{r} |
| pGalA3 | pTRKH2 + L. lactis MG1363 galA, Em^{r} |
| pRAF100 | pBS + 4 kpb EcoRI/HindIII fragment of |
| | L. raffinolactis ATCC43920 encoding aga, Ap^{r} |
| pRAF300 | pNZ123 + 4 kbp insertion of pRAF100 |
| pRAF301 | pNZ123 + 2.5 kbp aga amplicon from |
| | L. raffinolactis ATCC43920 |
| pRAF800 | Food-grade cloning vector, Mel+ |
| pRAF803 | pRAF800 + abiQ, AbiQ+ |
| pSRQ700 | Natural L. lactis plasmid, R/M+ |
| pSRQ800 | Natural L. lactis plasmid, AbiK+ |
| pSRQ900 | Natural L. lactis plasmid, AbiQ+ |
| pSRQ835 | pNC1 + replicon minimal de pSRQ800 |

^{a}Ap^{r}, ampicillin resistance;
Cm^{r}, chloramphenicol resistance;
Em^{r}, erythromycin resistance;
Mel, melibiose fermentation;
Abi, phage abortive infection mechanism;
R/M, type II restriction/modification system
Ts, thermosensitive replicon.
Quest Intl, Quest International, Rochester, Minnesota.

Cloning and Sequencing of aga From L. raffinolactis

The α-gal primers (Table 2) were used as a probe in Southern hybridizations to locate the alpha-galactosidase gene on specific restriction fragments of the L. raffinolactis genome. The primer was labeled using the DIG 3′-end oligonucleotide labeling kit (Roche Diagnostics). Pre-hybridization, hybridization, post-hybridization washes as well as detection by chemiluminescence were performed as suggested by the manufacturer (Roche Diagnostics). Restriction fragments of interest were extracted from 0.8% agarose gel after electrophoresis. DNA was recovered from the gel as described by Duplessis and Moineau (2001, Mol. Microbiol., 41:325-336). Relevant DNA fragments were cloned into pBS and the DNA sequences were determined on both strands using universal primers and the Tn1000 kit (Gold Biotechnology, St-Louis, Mo.). When needed, large amounts of E. coli plasmid DNA were isolated with the Qiagen Plasmid Maxi Kit (Chatsworth, Calif.). DNA sequencing was carried out by the DNA sequencing service at the Université Laval using an ABI Prism 3100 apparatus. Sequence analyses were performed using the Wisconsin Package software (version 10.2) of the Genetics Computer Group (GCG) (Devereux et al., 1984, Nuc. Acid Res., 253:270-272).

Construction of a L. lactis MG1363 galA Mutant

Two primers sets carrying terminal restriction sites were used to amplify by PCR the DNA regions upstream (galA5-galA6) and downstream (galA7-galA8) from galA of L. lactis MG1363. Both amplicons were digested with EcoRI, ligated together with T4 DNA ligase and re-amplified by PCR with primers galA5 and galA8. The resulting amplicon was digested with Xbal and Xhol and cloned into pGhost4 to generate pGalA2.

TABLE 2
Primers utilized

| | |
|---|---|
| Primer | Sequence (5′-3′) |

| | | |
|---|---|---|
| α-gal. | TTTGTTYTWGATGATGGWTTGTTTGGW | (SEQ ID NO:6) |
| abiQ1 | TCTAGATCTAGAACCCGTCCAAGGAAT | (SEQ ID NO:7) |
| | ATACAA | |
| abiQ2 | TCTAGATCTAGATGTTTCTAATCTAAA | (SEQ ID NO:8) |
| | TGACTGGT | |
| galA5 | TCTAGATCTAGACAAGGTCGCTCTGAT | (SEQ ID NO:9) |
| | ATTAG | |
| galA6 | GAATTCGAATTCGATCATGTCCTAGTG | (SEQ ID NO:10) |
| | CACCA | |
| galA7 | GAATTCGAATTCCTTTGTAGTCCCAGC | (SEQ ID NO:11) |
| | GGTCT | |
| galA8 | CTCGAGCTCGAGCCAATCAACAATGCG | (SEQ ID NO:12) |
| | AGCTC | |
| IB800.6 | ACATGACGATACCGCTACA | (SEQ ID NO:13) |
| IB800.8 | AATGCAAAAGACCGCTCTCA | (SEQ ID NO:14) |
| IB800.21 | TCTAGATCTAGAAGGGCTTGCCCTGAC | (SEQ ID NO:15) |
| | CGTCT | |
| IB800.23 | CTCGAGCTCGAGTTACACCTAACTCAT | (SEQ ID NO:16) |
| | CCGCA | |
| raf12 | TCTAGATCTAGAAGGGCTTGCCCTGAC | (SEQ ID NO:17) |
| | CGTCT | |
| raf13 | CTCGAGCTCGAGCCATCACCGAAGAGG | (SEQ ID NO:18) |
| | GCTGT | |
| raf39 | ATGAGTACCTCTCGTGACCA | (SEQ ID NO:19) |
| raf56 | GCTGGGATTAATCCCTTTGG | (SEQ ID NO:20) |
| raf63 | GAATTCGAATTCGTCTGTCGGTCTTC | (SEQ ID NO:21) |
| | AATATC | |

Homologous integration of pGalA2 into the chromosome of L. lactis MG1363 was achieved at 37° C. in presence of erythromycin. A pGalA2 integrant was selected and grown at 30° C. without selective pressure to favor excision and loss of the plasmid. Colonies were screened for erythromycin sensitivity and the presence of the mutated allele was confirmed by PCR using primers galA5 and galA8. For the complementation assay, the wild-type galA was amplified by PCR from MG1363 using primers galA5 and galA8 and the PCR product was cloned into pTRKH2 to construct pGalA3.

Isolation of the Minimal Replicon of Three L. lactis Plasmids

L. lactis plasmids pSRQ700, pSRQ800, and pSRQ900 (Boucher et al. 2001) were sub-cloned into the replicon-probe vector pNC1. The double-stranded Nested Deletion kit (Amersham Pharmacia Biotech, Baie d'Urfe, Quebec, Canada) was used to generate several deleted clones. These deletants were tested for their ability to replicate in L. lactis. The smallest replicative deletants originating from the three plasmids were sequenced both strands.

Construction of a Food-Grade Vector

The minimal replicon of pSRQ800 was amplify by PCR amplified using the primers IB800.21 and IB800.23 and pSRQ800 as the template. The aga from L. raffinolactis was also amplify by PCR using the primers raf12 and raf13 and L. raffinolactis total DNA. The two PCR products were digested with Xbal and Xhol, joined together and the ligation mixture was directly used to transform L. lactis MG1363 by electroporation. Cells were incubated for two hours for recuperation in the SM17MC medium supplemented with 0.5% melibiose. After recuperation, electroporated cells were inoculated into 10 ml of Mel-EL1 medium and incubated at room temperature until acidification which was manifested by the color change (from purple to yellow) of the pH indicator purple bromocresol. Then, cells were diluted in sterile peptonized water, plated on Mel-BCP plates and incubated for 24 h at 30° C. to recover melibiose-positive colonies. Plasmid DNA was recovered from the Mel+ colonies and sequenced.

Expression Profile of pRAF800

The transcription profiles of aga and repB encoded on pRAF800 were determined by RT-PCR. L. lactis was grown at 30° C. in 10 ml M17 supplemented with 0.5% of melibiose to an O.D.₆₀₀ of 0.2. The culture was pelleted and cell lysis was carried out in 100 μl TE containing 30 mg/ml lysozyme (Elbex, Quebec, Canada) at 37° C. for 10 min. Total RNA was then isolated using the RNeasy kit (Qiagen) as described by the manufacturer. The DNA was eliminated from the isolated RNA using RNAse-free DNAse in the presence of RNAse Inhibitor. Then, an additional RNeasy column was for RNA cleanup. All the reagents (RT buffer, DTT, dNTP, hexanucleotides, RNAse Inhibitor) except RT for the cDNA synthesis were mixed in microtubes. RNAse-free DNAse was added to the mixture for a second DNAse treatment at 37° C. for 30 min. The DNAse was heat inactivated at 75° C. (5 min.) and tubes were cooled to 4° C. The Expand™ Reverse Transcriptase (Roche Diagnostics) was added and the cDNA synthesis was performed essentially as recommended by the manufacturer. Two ul of the cDNA were used for PCR amplification using various primers combinations. The PCR products were fractionated by electrophoresis on a 0.8% agarose gel, stained with ethidium bromide and photographed under UV illumination with a Gel Documentation System (Bio-Rad™, Mississauga, ON).

Cloning of abiQ in pRAF800

The phage resistance gene abiQ was amplify by PCR using the primers abiQ1 and abiQ2 and pSRQ900 as the template. pRAF800 was digested with Xbal, dephosphorylated, and ligated to the Xbal digested abiQ amplicon. The ligation mixture was used to transform L. lactis MG1363 by electroporation, and Mel+ transformants were obtained as indicated above. Resistance to phage c2 was assessed as described previously (Moineau et al., 1992, Can. J. Microbiol., 38: 875-882).

Alpha-Galactosidase Assay

Strains were grown in 10 ml of M17 broth containing 0.5% melibiose to an OD₆₆₀ of 0.5-0.6. Cell pellets were washed twice in 50 mM sodium-phosphate (pH 7.0), and resuspended in 500 μl of the same buffer. Cells were disrupted at 4° C. by shaking on a vortex in presence of glass beads (106 μm and finer, Sigma) for by 3 bursts (3 minutes each followed by one minute rest on ice). Cell debris were removed by centrifugation and the supernatant (cell extract) was kept on ice until use in the enzyme assay which was completed within two hours. Protein concentrations were estimated using the Bio-Rad™ DC protein assay reagent (Mississauga, Ontario, Canada). Alpha-galactosidase activity was assayed at 30° C. and pH 7.0 using p-nitrophenyl-α-galactopyranoside (PNPG) as substrate. Essentially, 50 or 100 μl of cell extracts were added to the pre-warmed reaction mixture containing 250 μl of 3 mg/ml PNPG (Sigma) and enough Na.PO₄ 50 mM pH 7.0 to complete the volume at 3 ml. Aliquots of 900 μl were retrieved after 5, 10, and 15 minutes of incubation and added to 100 μl of chilled 1M Na₂CO₃. O.D.₄₂₀ was determined with a Beckman DU530 spectrophotometer and activity was calculated using PNPG ε₄₂₀=18300 M⁻¹.cm⁻¹.

Nucleotide Sequences Accession Numbers

The GenBank accession numbers assigned to the nucleotide sequences of Lactococcus plasmids pSRQ800, pSRQ900 and pRAF800 are U16027, U35629, and AF001314 respectively.

Results

Characterization of the Alpha-Galactosidase Locus of Lactococcus raffinolactis

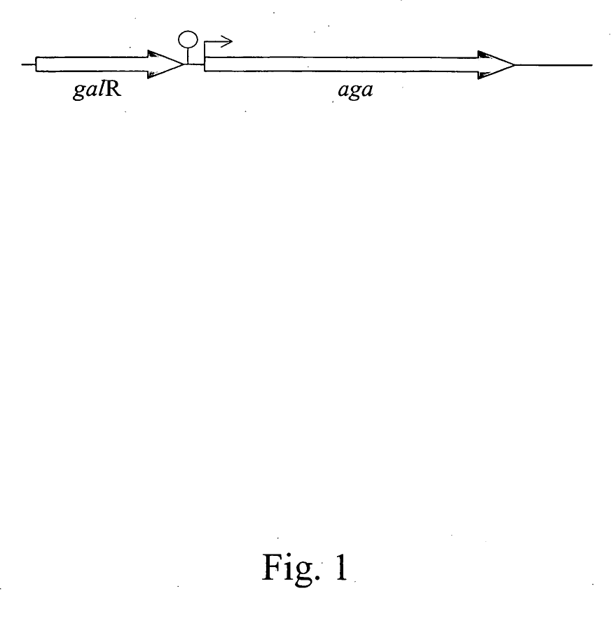

A stretch of conserved amino acids (FVLDDGWFG) was identified within bacterial alpha-galactosidases and used to design a degenerated oligonucleotide primer (α-gal, Table 2) based on lactococcal codon utilization preference. Using this primer as a probe in Southern hybridization assays, the alpha-galactosidase genetic determinant was located on a 4 kb EcoRI/HindIII genomic fragment (SEQ ID NO:3) of Lactococcus raffinolactis ATCC43920. This fragment (SEQ ID NO:3) was cloned into pBS (pRAF100), sequenced, and found to comprise two genes SEQ ID NO:4 and SEQ ID NO:5) encoding putative proteins similar to orthologues found in many Gram-positive bacteria (FIG. 1). Based on amino acid sequence similarities and conserved motifs, these two genes encode an alpha-galactosidase (Aga, 735 amino acids (SEQ ID NO:1)) and a transcriptional regulator (GalR, 245 aa, (SEQ ID NO:2)) from the Lacl/GalR family, respectively. The product of aga displays up to 54% identity with bacterial alpha-galactosidases, particularly Geobacillus stearothermophilus AgaN (390 identical amino acids out of 722), AgaB (376/730) and AgaA (372/730) (GenBank accession numbers AAD23585.1, AAG49421.1, and AAG49420.1). GalR is 34% identical to various transcriptional regulators including the galactose operon regulators from Lactobacillus casei (115/343) and Streptococcus thermophilus (112/340) (GenBank AAC19331.1, and AAD00092.1). An inverted repeat with the potential to form a stem loop structure was found in the aga-galR intergenic region and could act as an intrinsic terminator. A canonical promoter sequence (TTGACA-N₁₇-TATAAT) was found upstream of aga and a putative catabolite responsive element (CRE), involved in sugar metabolism regulation (Hueck et al., 1994), overlaps the −35 region. Consequently, the expression of aga is likely regulated through catabolite repression.

Cloning of aga in L. lactis

The 4 kb DNA fragment from L. raffinolactis was cloned into the lactococcal cloning vector pNZ123 (pRAF300) and transferred by electroporation into the laboratory strain L. lactis subsp. cremoris MG1363. The presence of pRAF300 conferred the ability to ferment melibiose to MG1363. This phenotype was easily observable since acidification due to sugar fermentation resulted in the formation of yellow colonies surrounded by a yellow halo on the purple background of BCP plates. On this medium, melibiose-negative cells formed smaller purple colonies on this medium. A 2.5 kb fragment containing only the aga gene was also amplify by PCR, cloned into pNZ123 (pRAF301) and transferred into the following five strains: L. lactis subsp. cremoris MG1363, L. lactis subsp. lactis IL1403 and SMQ-561, Streptococcus thermophilus SMQ-301 and Pediococcus acidilactici SMQ-249. The presence of pRAF301 was sufficient to confer the melibiose fermentation phenotype to all strains except S. thermophilus.

Identification of the Melibiose Carrier in L. lactis

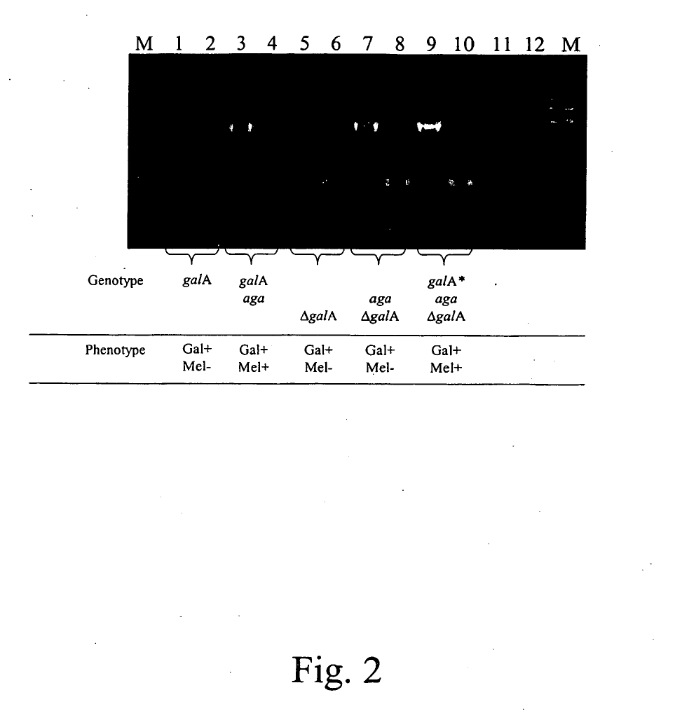

L. lactis subsp. cremoris MG1363 has a limited sugar fermentation pattern including acid production from galactose. As different galactosides can be imported through the same transporters (Poolman et al., 1996, Mol. Microbiol., 19:911-922), we hypothesized that the putative permease of the galactose operon GalA (Grossiord et al, 1998) might be the melibiose carrier in L. lactis MG1363. Using the suicide vector pGalA2 (see materials and methods for details), a L. lactis MG1363 galA deficient was constructed. After two homologous recombination events, 11 of the 24 Em^{s} clones tested contained a 600 pb truncated copy of galA instead of the 2 kbp wild-type allele (FIG. 2) In FIG. 2 the galA gene encoding the galactose operon permease of L. lactis MG1363 was inactivated and complemented with plasmid constructions containing aga (pRAF300) and the wild-type galA (pGalA3). Parental strain and mutants were analysed by PCR for their genotype (presence of the mutated allele of galA and aga) and for their phenotype (ability to produce acid from galactose and melibiose). Lanes 1 and 2, L. lactis MG1363; lanes 3 and 4, MG1363+pRAF300; lanes 5 and 6, MG1363 galA-deficient; lanes 7 and 8, MG1363 galA-deficient+pRAF300; lanes 9 and 10, MG1363 galA-deficient+pRAF300+pGalA3; lanes 11 and 12, negative controls. Odd numbers indicate the PCR amplification of aga using primers raf12 and raf13; even numbers show the PCR amplification of the disrupted galA with primers galA5 and galA8 (conditions of temperature and time elongation were maintained to amplify the disrupted allele only). aga: alpha-galactosidase; galA, galactose permease; ΔgalA, galA-deficient; galA*, galA supplied in trans. M, 1 kb DNA ladder (Invitrogen Life Technologies).

Surprisingly, all the galA mutants conserved their ability to produce acid from galactose. One of the galA⁻ mutant was selected and transformed with pRAF300. The 50 Cm^{r} transformants tested did not ferment melibiose. The wild type galA gene cloned into the coning vector pTRKH2 (pGalA3) was then introduced into L. lactis MG1363(ΔgalA, pRAF300) to complement the inactivation. All 24 Em^{r} Cm^{r} transformants tested were able to produce acid from melibiose, indicating that galA is require to obtain a Mel+ phenotype conferred by aga. All the transformants generated above (MG1363(ΔgalA), MG1363(ΔgalA, pRAF300), and MG1363(ΔgalA, pRAF300, pGalA3) were confirmed by plasmid profile, PCR amplification of aga and ΔgalA, and their sensitivity to phages c2 (c2 species) and p2 (936 species).

Isolation of Lactococcal Plasmid Replicons

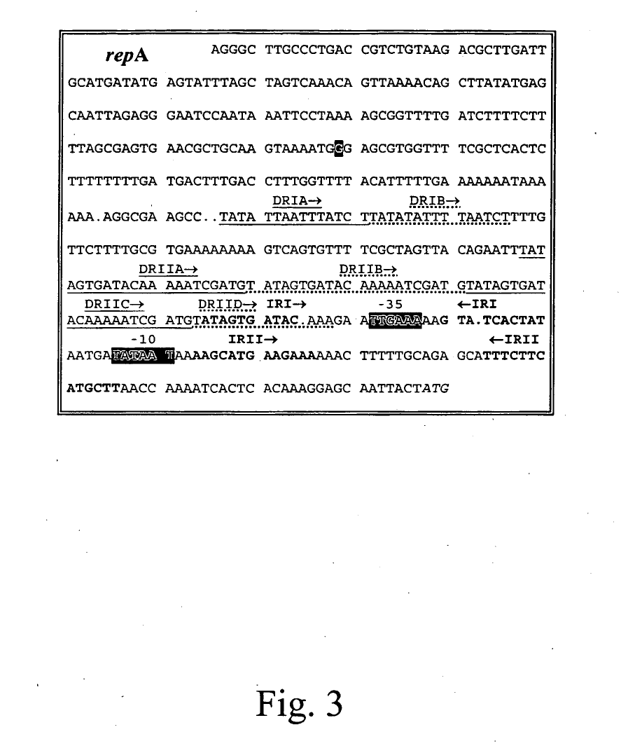

The minimal region essential for the maintenance of the natural lactococcal plasmid pSRQ800 was identified by operating successive deletions. For this plasmid, a DNA segment of 2212 bp encompassing positions 7196 through 1549 in the plasmid sequence was delimited and comprised a typical lactococcal theta replication module containing a replication origin (repA), and the gene encoding a replication initiator (repB). The replication origin include the AT-rich stretch, iterons and inverted repeats usually found in such genetic features (FIG. 3). The replicon of the natural L. lactis plasmid pSRQ800 was used to construct pRAF800. The nucleotide boxed in black is differs from pSRQ800 (T→G substitution). Direct repeats (DR) are underlined (continuous and discontinuous). Inverted repeats (IR) are indicated in bold character. The −35 and −10 boxes of the repB promoter are shaded. The repB start codon is italicized. The replicon of pSRQ800 was further limited from position −443 to +44 (from the repB coding sequence) to serve as the basis of a new plasmid vector. The replicons of two other L. lactis plasmids, namely pSRQ700 and pSRQ900, were also similarly delimited and could alternatively be used in the elaboration of novel genetic tools for lactic acid bacteria.

Construction of a Food-Grade Molecular Tool

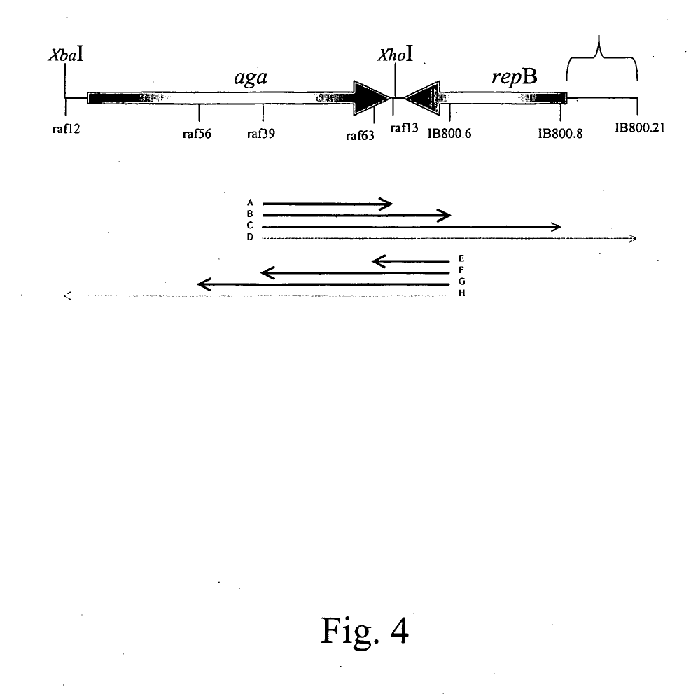

The aga of L. raffinolactis and the minimal replicon of L. lactis plasmid pSRQ800 were amplified by PCR and ligated together to form a functional cloning vector named pRAF800. The ligation mixture was electroporated into L. lactis MG1363 and after 4 days of incubation at room temperature in liquid EL1 media, Mel+ transformants were recovered on BCP plates. Plasmid DNA of a Mel+ transformant was isolated, digested, analyzed on agarose gel electrophoresis, and then sequenced on both strands. The 4245 bp constructed plasmid was named pRAF800 (FIG. 4). In FIG. 4, genes are identified by shaded arrows oriented to indicate the direction of transcription. aga, gene encoding alpha-galactosidase; repB, gene encoding the replication initiator. Plasmid replication origin is located between repB and the Xbal site. The position of primers used for RT/PCR is indicated on the plasmid map. This novel plasmid differed from the parental DNA segments in four locations. The first difference is a non-conservative A/G substitution causing a T/A amino acid change at position 227 of the alpha-galactosidase enzyme. A second A/G substitution is found at position 2424, immediately downstream the aga coding sequence. A frameshift was also found within primer raf13 sequence, suggesting a likely error in the primer sequence itself. Finally, a T/G substitution is localized 358 nt upstream of the repB start codon, in the replication origin region (FIG. 3). These variations did not affect the plasmid functionality.

Cloning of a Phage Resistance Mechanism into pRAF800 and its Transfer into an Industrial L. lactis Strain

The phage abortive infection mechanism AbiQ from pSRQ900 (Emond et al., 1998, Appl. Environ. Microbiol., 64:4748-4756) was cloned into pRAF800. The phage resistance determinant was obtained by PCR amplification and inserted into the unique Xbal site of pRAF800 to generate pRAF803. The recombinant vector was first obtained in L. lactis MG1363 that became resistant to phage c2. Both plasmids pRAF800 and pRAF803 were then transferred by electroporation into the industrial L. lactis subsp. cremoris strain SMQ-741. Because this strain did not grow well in EL1 medium, the post-transformation enrichment was performed in liquid BCP media containing melibiose. All 48 Mel+ colonies tested contained pRAF803 and were resistant to phage Q37 while Mel− colonies did not contained pRAF803 and were sensitive to phage Q37.

Expression Profile of pRAF800 in the Industrial Strain SMQ-741

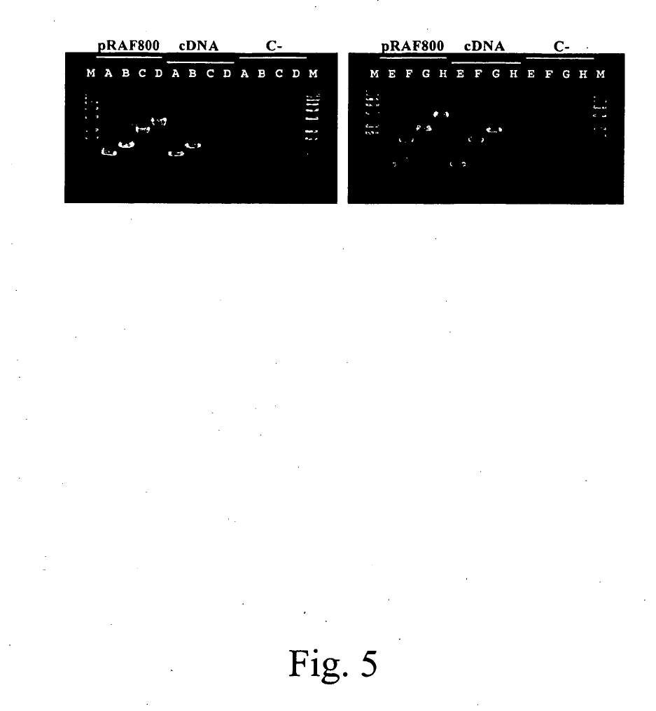

The transcription profiles of aga and repB were determined by RT-PCR using RNA isolated from L. lactis SMQ-741+pRAF800. Two overlapping transcripts were observed (FIG. 0.5). IN FIG. 5, the RNA was isolated from SMQ-741 cells transformed with pRAF800 and grown in the presence of melibiose. RT-PCR was used to map the aga and repB transcripts. PCR products were separated by electrophoresis on 0.8% agarose gel and stained with ethidium bromide. The targeted transcripts are identified by arrows A-H (indicating the direction of transcription), as seen on the gels. PCR products are aligned with their corresponding start and stop positions on the plasmid map. Bold line, transcript detected; normal line, transcript weakly detected; gray line, transcript not detected. M, 1 kb DNA ladder (Invitrogen Life Technologies).

The repB transcript overlaps most of the aga sequence and is likely to terminate at the inverted repeat located immediately upstream of aga (FIG. 1). The aga transcript also extends beyond repB and is suspected to end at one of the multiple inverted repeats located in this gene. A weak signal obtained with primer IB800.8 would point toward non-specific transcriptional termination at multiple sites. Taken altogether, these data indicated that the Xhol site region is transcribed in both directions from the two promoters of aga and repB. At the opposite, the Xbal site region did not appear to be transcribed from any of the two promoters located on pRAF800.

Alpha-Galactosidase Activity in L. lactis

The activity of alpha-galactosidase was measured in cell extracts from L. lactis SMQ-741+pRAF800 and grown at 30° C. in presence of various sugars (Table 3). Activity was measured under conditions where the rate of reaction was constant with the time of incubation and proportional with the enzyme concentration. The results summarized in Table 3 indicate that the alpha-galactosidase activity was induced 4 to 5-fold by galactose and melibiose but not by glucose or lactose. As no alpha-galactosidase activity could be detected with the parental strain SMQ-741, aga is clearly responsible of this activity in L. lactis. The enzymatic activity measured in L. lactis grown in melibiose was comparable to the activity obtained with L. raffinolactis ATCC 43920 grown in the same sugar.

TABLE 3
Alpha-galactosidase activity in L. lactis SMQ-741
transformed with pRAF800 and grown in the presence of
various sugars

| | Activity^{ab} |
|---|---|
| | (nmol of p-nitrophenol formed/mg of |
| Sugar | protein/min) |
| glucose | 74.8 ± 16.2 |
| galactose | 332.2 ± 27.3 |
| lactose | 67.8 ± 15.5 |
| melibiose | 397.4 ± 31.6 |

^{a}Values are the means ± standard deviations of twelve measurements performed with two extract quantities in two independent experiments.
^{b}Alpha-galactosidase activity was not detected in the parental strain SMQ-741.

In conclusion, a novel small plasmid vector was constructed based on a L. lactis theta replication module and a L. raffinolactis aga gene encoding an alpha-galactosidase as a selection marker. Constituted exclusively of lactococcal DNA and exempt of antibiotic resistance genes, the proposed vector should therefore be appropriate for a safe use in the food industry. The Mel+ phenotype conferred by L. raffinolactis aga gene emerged as a convenient dominant selection marker operating with a practical melibiose-containing medium. Lactococcal alpha-galactosidases represent new molecular tools for the genetic modification of lactococci and other lactic acid bacteria that could be exploited for research purposes as well as food related applications.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. An alpha-galactosidase protein isolated from food grade lactic acid bacteria, comprising amino acid sequence as set forth In SEQ ID NO:1, or analogs thereof having at least 80% or homology, and having alpha-galactosidase activity.

2. An alpha-galactosidase regulator protein isolated from food grade lactic acid bacteria, comprising amino acid sequence as set forth SEQ ID NO:2, or analogs thereof having at least 80% or homology, and having an alpha-galactosidase regulator activity.

3. An isolated DNA sequence as set forth in SEQ ID NO:3 encoding for an alpha-galactosidase protein and an alpha-galactosidase regulator.

4. An Isolated DNA sequence as set forth in SEQ ID NO:4 encoding for an alpha-galactosidase protein.

5. An isolated DNA sequences as set forth in SEQ ID NO:5 encoding for an alpha-galactosidase regulator.

6. An isolated DNA sequence from Lactotoccus raffinolactis selected from the group consisting of SEQ ID NO:3, SEQ ID NO:4, and SEQ ID NO:5.

7. A vector suitable for transforming a host cell, said vector comprising;
at least one DNA sequence as defined in claim 6;
a suitable promoter allowing expression of said DNA sequence in said host cell;
wherein said DNA sequence encodes for a protein having alpha-galactosidase activity.

8. The vector as claimed in claim 7, comprising a DNA sequence as set for in SEQ ID NO:4 and coding for an alpha-galactosidase.

9. The vector as claimed in claim 7, wherein said protein is expressed in said host cell as a selectable marker.

10. The vector as claimed in claim 7, which is a food-grade vector.

11. A host cell transformed with vector as defined in to claim 7.

12. The host cell as claimed in claim 11 which is a food-grade host cell.

13. The host cell as claimed in claim 11, comprising a host cell selected from the group consisting of an animal cell, a yeast, and a bacteria.

14. The host cell as claimed in claim 13, wherein said bacteria is selected from the group consisting of Lactococcus, Streptococcus, Lactobacillus, Leuconostocs, Pediococcus, Bifidobacterium, Oenococcus, and Proplonibacterium.

15. Use or a DNA sequence as defined in claims 3 to 5 in the preparation of a vector allowing expression of at least one of an alpha-galactosidase protein or an alpha-galactosidase regulator.


SEQUENCE LISTING (21 sequences)

SEQ ID NO: 1 - protein, 735 aa, Lactococcus
  PEPTIDE (1)...(735)
MTLITFDENNKIFHLSNTSISYLIGIEKESYLSHLYFGKVIKTYHAGRKYPAMNRSFSPNPDGMPLNTRDFSLDVISQEFPSYGHGDFRNPAVQIKQTNGSSITEFVYDSYEIISGKPILDGLPATYVGGDEEAETLVITLIDKLLNLKLKLSYTIYAQRNVIARNALLENNGMAPVVIEKLASLSVDLPEQDLELISLPGRHVKEREIERQTIQRGTRIIDSKRGTSSHQSNPFIAIVEPKTDEFTGTAIGLTLVYSGNHEMLVERDQFSQTRVMAGINPFGFEWELESDASFQSPEALLVYSDQGLNGMSQTFHDLLQNRLARGQYRQAERPILINNWEATYFDFDTDKIKKIVDSAADLGIELFVLDDGWFGKRDDDTSGLGDWFENTEKLKGGLKGIADYVHQKNMTFGLWFEPEMVNADSDLFRQHPDYALQIPGRSMSTSRDQYVLDFSRKEVRETITAQMRAILDTIDIDYIKWDMNRNLTEVYSATASAAHQGEVFHRYVLGLYEMLEELTTDYDHILWEGCSGGGGRFDAGFLHYMPQSWTSDNTDAVERLDIQYGTSLVYPISSMGAHVSAVPNHQTYRETGLEIRGDVAMSGVFGYELNLQDMTQEEKAVVLEQVAFYKTHRKLLQYGKFHRLLSPFESDQTAWLFVNGDQSQAIGFYFRKYAESAGPLRTLKFTGLAPEKTYQVNGDAIYGGDELMSVGLHIYPFLVGDYQSRKFVINEVKSN

SEQ ID NO: 2 - protein, 345 aa, Lactococcus
  PEPTIDE (1)...(345)
MASIREIAKLAGVSPATVSRVLNADETMSVSPATRTRIIKVANQLNYHKVENLGPKSPKQSYKLSIAVIKTHSSKRENDDPYFRLIQEGIALEAGNWNFRLETLKLGEVSLEQLAQFGAVLTIGAFTDETLADIYKVNQNLIVVDNHFASSRYDLVHTDFAKQTEQVLDYLYEQNHRQIAFIGGEIRTVDLNGQNQYLLADVRTTAYENWMTIHGLSDNIQIKTGDWTMAFALNATNELVKSSGDQLPTAIISASDPMSIGIYRALQLKNIDIPETISVFSFDDIEMAGFMSPPLSTVHIDSLEIGRVAVRLAKERISDGRKTALRVEVASEIIVRDSVRKNKLS

SEQ ID NO: 3 - DNA, 3246 bp, Lactococcus
  gene (1)...(3246)
atggcgagtattagagaaattgcaaaattagcaggtgtttcgcctgctacggtatcacgagtcttaaatgcggatgaaaccatgagtgtttcaccagcaacccgcaccagaattattaaagtagccaaccagcttaattatcacaaagtggaaaatttagggcctaaatctccaaaacaatcttataaattatcgattgctgtgatcaagacgcattcatccaaacgtgaaaatgatgatccttattttcgtttgattcaggaaggtatcgcacttgaagcgggtaattggaattttagacttgaaacgctgaagttaggggaagtgagccttgagcagcttgcgcaatttggagcggttcttacgattggtgcttttacagatgagacgctggcagatatttacaaagtcaatcaaaatctcattgtcgttgataatcatttcgcaagctctcgttatgatctcgttcataccgactttgccaaacaaacagagcaagtcttggattatctttatgaacaaaatcatcgacaaatcgccttcattggtggtgaaattagaacggttgatttgaatgggcagaatcaatacttattagcagacgttaggactactgcctatgaaaactggatgaccatccatggtttgtcagataatatccaaattaaaacgggtgattggacgatggcatttgctttaaatgccacaaatgagttagttaaatcgtctggcgatcagttgccaacagccattatttccgctagtgatcccatgagtatcgggatttatcgtgcgttgcagctgaaaaacatcgacattccagaaactatttctgtatttagttttgatgatattgagatggcaggctttatgtcaccgccacttagtacagtccatattgatagtctggagattggacgtgttgccgttagattagctaaagaacgtatttcagacggtcgaaagactgccctacgtgttgaagttgcctcagagattatcgtccgtgatagcgttcgaaaaaacaaactttcataaatgacactaatcacatttgatgaaaacaacaaaatttttcacctctcaaacacatccatttcttatctcatcggcattgaaaaagaaagttatctgagtcatctttatttcggtaaagtcattaaaacttatcatgctggtcggaaatatccagccatgaatcgtagtttctcgccaaatccggatgggatgccgcttaatacacgtgatttttcattggatgtcatctcacaagaatttccaagttatggtcacggcgatttccgaaatcctgctgttcaaattaagcaaacaaatggctcatcaatcactgaatttgtttacgatagctatgagattatttctggtaaacctatacttgatggtttaccagcgacttatgtaggaggtgatgaagaagctgaaacacttgtcatcaccttgattgataaactactcaacctcaaattaaaactttcgtacacgatttatgcacaacgcaatgtgattgcgcgtaatgccttgcttgaaaataatgggatggcaccagttgtcattgaaaagttagcgagtttatcagttgatttaccagagcaagacttggagctgattagtttgcctggacgacatgtcaaagaacgtgaaattgaacgtcagacgattcaacgtggcacacgaatcatcgacagcaaacgtggcacgtcaagccaccaatctaatccatttattgcgatcgttgaacccaaaacagacgaatttacagggacagcaattggtttaactttagtctatagtggcaatcatgaaatgttggttgaacgagatcaattttcacagacgcgagtcatggctgggattaatccctttggttttgaatgggaacttgagagtgatgcgtctttccaatcacctgaagccctgttagtttattctgatcaaggacttaatggcatgagccaaacctttcatgacttacttcaaaaccgattggcgcgtggacaataccgtcaggcggagcgcccaatcctcatcaataactgggaagcgacttattttgactttgatacggataaaatcaagaaaattgttgatagtgccgctgatcttggaattgaacttttcgttttagatgatggctggtttggtaaacgtgatgatgatacatcaggtttgggagattggtttgaaaatacagagaagctaaaaggtggactcaaaggcatcgctgactatgtgcatcaaaaaaatatgacctttggcctttggtttgaacctgaaatggttaatgcggatagtgacctatttcgtcaacatcctgattacgcccttcaaataccagggcgttctatgagtacctctcgtgaccaatacgtgctagatttttcacgcaaagaagttcgcgagacaattacagcgcaaatgcgggctattcttgatacgattgacattgactatatcaaatgggacatgaaccgtaacttgacggaagtttattcagcgacagcaagtgcagcgcaccaaggtgaagtcttccatcgatacgttttaggactctatgagatgctagaagaactgacgacggactatgatcatatcctttgggaaggctgctcaggtggtggcggtaggttcgatgctggatttttacattacatgccacaaagttggacgagcgataatacagatgccgttgaacgcttagatattcagtatggcacgagcctagtctaccccatttcttcgatgggtgcccatgtatcagcagtgcccaatcaccaaacatatcgggaaacaggcttagagattcgaggtgatgtcgccatgagcggggtctttggttatgaactgaatcttcaagacatgacacaagaagaaaaagcagtcgttcttgaacaggttgctttctacaaaacacatcgtaaactcttgcagtatggaaaattccatcgtctcttatcaccatttgaatcagatcaaacagcatggctttttgtcaatggtgatcagtcacaagccatcggtttctattttagaaaatatgcagaatcagctggtccccttcggacacttaaattcacaggacttgcacctgagaaaacttatcaagtcaacggtgatgccatatatggtggtgatgaattaatgtctgtcggtcttcatatctatcctttcctagttggagattatcaaagtcgcaaatttgtcataaatgaggtcaagtcaaattag

SEQ ID NO: 4 - DNA, 1038 bp, Lactococcus
  gene (1)...(1038)
atggcgagtattagagaaattgcaaaattagcaggtgtttcgcctgctacggtatcacgagtcttaaatgcggatgaaaccatgagtgtttcaccagcaacccgcaccagaattattaaagtagccaaccagcttaattatcacaaagtggaaaatttagggcctaaatctccaaaacaatcttataaattatcgattgctgtgatcaagacgcattcatccaaacgtgaaaatgatgatccttattttcgtttgattcaggaaggtatcgcacttgaagcgggtaattggaattttagacttgaaacgctgaagttaggggaagtgagccttgagcagcttgcgcaatttggagcggttcttacgattggtgcttttacagatgagacgctggcagatatttacaaagtcaatcaaaatctcattgtcgttgataatcatttcgcaagctctcgttatgatctcgttcataccgactttgccaaacaaacagagcaagtcttggattatctttatgaacaaaatcatcgacaaatcgccttcattggtggtgaaattagaacggttgatttgaatgggcagaatcaatacttattagcagacgttaggactactgcctatgaaaactggatgaccatccatggtttgtcagataatatccaaattaaaacgggtgattggacgatggcatttgctttaaatgccacaaatgagttagttaaatcgtctggcgatcagttgccaacagccattatttccgctagtgatcccatgagtatcgggatttatcgtgcgttgcagctgaaaaacatcgacattccagaaactatttctgtatttagttttgatgatattgagatggcaggctttatgtcaccgccacttagtacagtccatattgatagtctggagattggacgtgttgccgttagattagctaaagaacgtatttcagacggtcgaaagactgccctacgtgttgaagttgcctcagagattatcgtccgtgatagcgttcgaaaaaacaaactttcataa

SEQ ID NO: 5 - DNA, 2208 bp, Lactococcus
  gene (1)...(2208)
atgacactaatcacatttgatgaaaacaacaaaatttttcacctctcaaacacatccatttcttatctcatcggcattgaaaaagaaagttatctgagtcatctttatttcggtaaagtcattaaaacttatcatgctggtcggaaatatccagccatgaatcgtagtttctcgccaaatccggatgggatgccgcttaatacacgtgatttttcattggatgtcatctcacaagaatttccaagttatggtcacggcgatttccgaaatcctgctgttcaaattaagcaaacaaatggctcatcaatcactgaatttgtttacgatagctatgagattatttctggtaaacctatacttgatggtttaccagcgacttatgtaggaggtgatgaagaagctgaaacacttgtcatcaccttgattgataaactactcaacctcaaattaaaactttcgtacacgatttatgcacaacgcaatgtgattgcgcgtaatgccttgcttgaaaataatgggatggcaccagttgtcattgaaaagttagcgagtttatcagttgatttaccagagcaagacttggagctgattagtttgcctggacgacatgtcaaagaacgtgaaattgaacgtcagacgattcaacgtggcacacgaatcatcgacagcaaacgtggcacgtcaagccaccaatctaatccatttattgcgatcgttgaacccaaaacagacgaatttacagggacagcaattggtttaactttagtctatagtggcaatcatgaaatgttggttgaacgagatcaattttcacagacgcgagtcatggctgggattaatccctttggttttgaatgggaacttgagagtgatgcgtctttccaatcacctgaagccctgttagtttattctgatcaaggacttaatggcatgagccaaacctttcatgacttacttcaaaaccgattggcgcgtggacaataccgtcaggcggagcgcccaatcctcatcaataactgggaagcgacttattttgactttgatacggataaaatcaagaaaattgttgatagtgccgctgatcttggaattgaacttttcgttttagatgatggctggtttggtaaacgtgatgatgatacatcaggtttgggagattggtttgaaaatacagagaagctaaaaggtggactcaaaggcatcgctgactatgtgcatcaaaaaaatatgacctttggcctttggtttgaacctgaaatggttaatgcggatagtgacctatttcgtcaacatcctgattacgcccttcaaataccagggcgttctatgagtacctctcgtgaccaatacgtgctagatttttcacgcaaagaagttcgcgagacaattacagcgcaaatgcgggctattcttgatacgattgacattgactatatcaaatgggacatgaaccgtaacttgacggaagtttattcagcgacagcaagtgcagcgcaccaaggtgaagtcttccatcgatacgttttaggactctatgagatgctagaagaactgacgacggactatgatcatatcctttgggaaggctgctcaggtggtggcggtaggttcgatgctggatttttacattacatgccacaaagttggacgagcgataatacagatgccgttgaacgcttagatattcagtatggcacgagcctagtctaccccatttcttcgatgggtgcccatgtatcagcagtgcccaatcaccaaacatatcgggaaacaggcttagagattcgaggtgatgtcgccatgagcggggtctttggttatgaactgaatcttcaagacatgacacaagaagaaaaagcagtcgttcttgaacaggttgctttctacaaaacacatcgtaaactcttgcagtatggaaaattccatcgtctcttatcaccatttgaatcagatcaaacagcatggctttttgtcaatggtgatcagtcacaagccatcggtttctattttagaaaatatgcagaatcagctggtccccttcggacacttaaattcacaggacttgcacctgagaaaacttatcaagtcaacggtgatgccatatatggtggtgatgaattaatgtctgtcggtcttcatatctatcctttcctagttggagattatcaaagtcgcaaatttgtcataaatgaggtcaagtcaaattag

SEQ ID NO: 6 - DNA, 27 bp, Lactococcus
  alpha-gal
tttgttytwgatgatggwttgtttggw

SEQ ID NO: 7 - DNA, 33 bp, Lactococcus
  abiQ1
tctagatctagaacccgtccaaggaatatacaa

SEQ ID NO: 8 - DNA, 35 bp, Lactococcus
  abiQ2
tctagatctagatgtttctaatctaaatgactggt

SEQ ID NO: 9 - DNA, 32 bp, Lactococcus
  galA5
tctagatctagacaaggtcgctctgatattag

SEQ ID NO: 10 - DNA, 32 bp, Lactococcus
  galA6
gaattcgaattcgatcatgtcctagtgcacca

SEQ ID NO: 11 - DNA, 32 bp, Lactococcus
  galA7
gaattcgaattcctttgtagtcccagcggtct

SEQ ID NO: 12 - DNA, 32 bp, Lactococcus
  galA8
ctcgagctcgagccaatcaacaatgcgagctc

SEQ ID NO: 13 - DNA, 19 bp, Lactococcus
  IB800.6
acatgacgataccgctaca

SEQ ID NO: 14 - DNA, 20 bp, Lactococcus
  IB800.8
aatgcaaaagaccgctctca

SEQ ID NO: 15 - DNA, 32 bp, Lactococcus
  IB800.21
tctagatctagaagggcttgccctgaccgtct

SEQ ID NO: 16 - DNA, 32 bp, Lactococcus
  IB800.23
ctcgagctcgagttacacctaactcatccgca

SEQ ID NO: 17 - DNA, 32 bp, Lactococcus; identical to SEQ ID NO: 15
  raf12

SEQ ID NO: 18 - DNA, 32 bp, Lactococcus
  raf13
ctcgagctcgagccatcaccgaagagggctgt

SEQ ID NO: 19 - DNA, 20 bp, Lactococcus
  raf39
atgagtacctctcgtgacca

SEQ ID NO: 20 - DNA, 20 bp, Lactococcus
  raf56
gctgggattaatccctttgg

SEQ ID NO: 21 - DNA, 32 bp, Lactotoccus
  raf63
gaattcgaattcgtctgtcggtcttcaagatc